(19) United States
(12) Patent Application Publication
(10) Pub. No.: US 2025/0009515 A1
(43) Pub. Date: Jan. 9, 2025

(54) PIEZOELECTRIC NON-RETURN VALVE

(71) Applicant: Boston Scientific Scimed, Inc., Maple Grove, MN (US)

(72) Inventors: Daragh Nolan, Youghal (IE); Richard Percy, Leamlara (IE); Thomas Sinnott, Enniscorthy (IE); Eduardo Marcos Larangeira, Clonmel (IE)

(21) Appl. No.: 18/760,693

(22) Filed: Jul. 1, 2024

Related U.S. Application Data
(60) Provisional application No. 63/512,150, filed on Jul. 6, 2023.

(51) Int. Cl. A61F 2/26; F04B 43/04; F16K 31/00

(52) U.S. Cl. CPC A61F 2/26; F04B 43/046; F16K 31/005

(57) ABSTRACT

An implantable fluid operated device includes a fluid control system to transfer fluid between a fluid reservoir and an inflatable member. The fluid control system includes at least one fluid control device including at least one pump and at least one valve, or at least one combination pump and valve device. The at least one fluid control device includes an auxiliary fluid control device that maintains a closed state of the valve in response to a fluctuation in pressure, or a pressure spike that would otherwise cause an unintentional opening of the fluid control device, and unintentional flow of fluid through the fluid control device.

### CROSS-REFERENCE TO RELATED APPLICATION

This application claims priority to U.S. Provisional Patent Application No. 63/512,150, filed on Jul. 6, 2023, entitled “PIEZOELECTRIC NON-RETURN VALVE”, the disclosure of which is incorporated by reference herein in its entirety.

### TECHNICAL FIELD

This disclosure relates generally to bodily implants, and more specifically to bodily implants including a fluid control system having one or more pumps and/or valves including a piezoelectric actuator.

### BACKGROUND

Active implantable fluid operated inflatable devices can include one or more pumps that regulate a flow of fluid between different portions of the implantable device. One or more valves can be positioned within fluid passageways of the device to direct and control the flow of fluid to achieve inflation, deflation, pressurization, depressurization, activation, deactivation and the like of different fluid filled components of the device. In some implantable fluid operated devices, an implantable pumping device may be manually operated by the user to provide for the transfer of fluid between a reservoir and the fluid filled implant components of the device. In some situations, manual operation of the pumping device may make it difficult to achieve consistent inflation, deflation, pressurization, depressurization, activation, deactivation and the like of the fluid filled implant components. Inconsistent inflation, deflation, pressurization, depressurization, activation and/or deactivation of the fluid filled implant device(s) may adversely affect patient comfort, efficacy of the device, and the overall patient experience. Some implantable fluid operated devices include an electronic control system including an electronically controlled manifold providing for the transfer of fluid within the implantable fluid operated device. The use of the electronic control system may provide for more accurate actuation and control of the flow of fluid between components of the inflatable device, thus improving performance and efficacy of the device, as well as patient comfort and safety. Consistent inflation, deflation, pressurization, depressurization, activation, deactivation and the like of the fluid filled implant components may rely on accurate flow control through the pumps and/or valves within a manifold of the electronic control system. Fluctuations in pressure may be experienced within the manifold due to, for example, movement of the user, falls, and the like. A system and method for maintaining set positions of various components of the pumps and/or valves in the manifold in the event of fluctuations in pressure, pressure spikes and the like, may provide for consistent flow of fluid through the manifold, and for consistent, accurate control of the inflation, deflation, pressurization, depressurization, activation, deactivation and the like of the fluid filled components of the implantable fluid operated inflatable device.

### SUMMARY

In some aspects, the techniques described herein relate to an implantable fluid operated inflatable device, including a fluid control system configured to control fluid flow between a fluid reservoir and an inflatable member, the fluid control system including at least one fluid passageway defined within a housing; and a fluidic component positioned in the at least one fluid passageway and configured to provide for a flow of fluid in a first flow direction, and to restrict a flow of fluid in a second flow direction, the fluidic component including a fluid chamber defined between a base plate and a deformable diaphragm; and an auxiliary fluid control device provided at one of an inlet into the fluid chamber or an outlet of the fluid chamber, wherein in an open state of the fluidic component, the auxiliary fluid control device is in an open state such that inlet, the fluid chamber, and the outlet provide for fluid flow through the fluidic component in the first flow direction, and in a closed state of the fluidic component, the auxiliary fluid control device is configured to close one of the inlet or the outlet of the fluid chamber in response to a force applied in the second flow direction, to maintain the closed state of the fluidic component.

In some implementations, the auxiliary fluid control device includes a spring check valve positioned in the outlet, wherein in the open state of the fluidic component, the spring check valve is opened in response to a force of fluid, flowing from the fluid chamber toward the outlet, exerted on a disc portion of the spring check valve, and in the closed state of the fluidic component, the spring check valve is closed against a foil positioned between the spring check valve and the outlet in response to a force of fluid, flowing from the outlet toward the fluid chamber, exerted on the disc portion of the spring check valve.

In some implementations, a dimension of the disc portion of the spring check valve is greater than a corresponding dimension of an opening formed in the foil, such that in the closed state the force exerted on the disc portion maintains the spring check valve in the closed state.

In some implementations, the auxiliary fluid control device includes a seal positioned on the base plate, at a position corresponding to the inlet, the seal including a body portion; a first flange portion at a first end portion of the body portion; and a second flange portion at a second end portion of the body portion, wherein the first flange portion is coupled to the base plate, with an opening extending through the body portion aligned with the inlet, and the second flange portion is positioned in the fluid chamber; in the open state of the fluidic component, fluid flows from the inlet, through a space in the fluid chamber between the second flange portion and the diaphragm, and out of the fluidic component through the outlet; and in the closed state of the fluidic component, the second flange portion is positioned against the diaphragm and forms a seal with the diaphragm, and the second flange portion is configured to deform so as to maintain the seal with the diaphragm in response to the force of fluid from the outlet exerted on the diaphragm and a corresponding deformation of the diaphragm.

In some implementations, the auxiliary fluid control device includes an umbrella valve positioned in inlet, the umbrella valve including a body portion positioned in the inlet; a first flange portion at a first end portion of the body portion; at least one opening formed in the first flange portion, aligned with the inlet; and a second flange portion at a second end portion of the body portion, positioned in the fluid chamber, wherein in the open state of the fluidic component, the umbrella valve is in a first position in the inlet, in which the second flange portion is spaced apart from a first side portion of the base plate to provide for fluid communication between the inlet and the fluid chamber, and the first flange portion is positioned at a second side portion of the base plate, with the at least one opening in the first flange portion aligned with the inlet, such that fluid flows through the at least one opening, through the inlet into the fluid chamber and out of the fluidic component through the outlet; and in the closed state of the fluidic component, the umbrella valve is in a second position in the inlet, in which the second flange portion is positioned on the first side portion of the base plate, extending across the inlet in response to a force of fluid from the outlet so as to maintain a closed state of the inlet and the closed state of the fluidic component.

In some implementations, the auxiliary fluid control device includes a flap seal valve positioned in the inlet, the flap seal valve including a body portion; a base portion at a first end portion of the body portion, the base portion being fixed in the inlet, with an opening extending through the base portion to provide for fluid communication with the fluid chamber; and a flap portion at a second end portion of the body portion, wherein in the open state of the fluidic component, fluid flows through the opening in the base portion and into fluid chamber, and out of the fluidic component through the outlet, and in the closed state of the fluidic component, the flap portion is configured to deform so as to close the opening in the base portion in response to a force of fluid from the outlet toward the fluid chamber.

In some implementations, the fluidic component is a piezoelectric valve device, including a piezoelectric element coupled to the deformable diaphragm; at least one electrode coupled to the piezoelectric element, wherein the deformable diaphragm is configured to deform in response to voltage applied to the piezoelectric element to move between the open state and the closed state of the fluidic component; a first fluid passageway and a first opening formed in the base plate, wherein the first fluid passageway and the first opening define the inlet into the fluid chamber; and a second fluid passageway and a second opening formed in the base plate, wherein the second fluid passageway and the second opening define the outlet of the fluid chamber.

In some implementations, the first flow direction provides for the flow of fluid from the fluid reservoir to the inflatable member, and the second flow direction provides for the flow of fluid from the inflatable member to the fluid reservoir.

In some implementations, the inlet is defined by a first opening in the base plate providing for fluid communication between a first fluid passageway of the fluidic component and the fluid chamber; and the outlet is defined by a second opening in the base plate providing for fluid communication between the fluid chamber and a second fluid passageway of the fluidic component.

In some implementations, the fluidic component is one of a valve device, a pump device, or a combined pump and valve device.

In some aspects, the techniques described herein relate to a fluid control system for an implantable fluid operated inflatable device, including a housing; at least one fluid passageway defined within the housing; and a valve device positioned in the at least one fluid passageway, the valve device including a base plate; a diaphragm coupled to the base plate; a fluid chamber defined between the base plate and the diaphragm; a first opening formed in the base plate, the first opening connecting the fluid chamber to a first fluid passageway; a second opening formed in the base plate, the second opening connecting the fluid chamber to a second fluid passageway; and a fluid control device provided at the one of the first opening or the second opening, wherein in an open state of the valve device, the first fluid passageway, the fluid chamber, and the second fluid passageway guides fluid through the valve device in a first flow direction, and in a closed state of the valve device, the fluid control device is configured to close the one of the first fluid passageway or the second fluid passageway in response to a force applied in a second flow direction, opposite the first flow direction, to maintain the closed state of the valve device.

In some implementations, the fluid control device includes a spring check valve positioned in the second fluid passageway, wherein in the open state of the valve device, the spring check valve is opened in response to a force of fluid flowing from the fluid chamber to the second fluid passageway, and in the closed state of the valve device, the spring check valve is closed against the diaphragm in response to a force of fluid flowing from the second fluid passageway toward the fluid chamber, wherein a dimension of a disc portion of the spring check valve is greater than a corresponding dimension of the second opening in the base plate, such that in the closed state the force exerted on the disc portion by fluid flowing from the second fluid passageway toward the fluid chamber maintains the spring check valve in a closed state against the second opening in the base plate.

In some implementations, the fluid control device includes a seal positioned on the base plate, at a position corresponding to the first opening in the base plate, wherein in the open state of the valve device, fluid flows through the valve device from the first fluid passageway, through a space in the fluid chamber between the seal and the diaphragm, and out of the valve device through the second fluid passageway, and in the closed state of the valve device, a deformable portion of the seal deforms in response to a force of fluid flowing from the second fluid passageway toward the fluid chamber to block a flow of fluid from the fluid chamber into the first fluid passageway.

In implementations, the deformable portion of the seal includes one of a flange portion of the seal that deforms in response to the force, to maintain a seal with the diaphragm, or a flap portion of the seal that deforms in response to the force, to close an opening in a base portion of the seal positioned in the first fluid passageway.

In some implementations, the fluid control device includes an umbrella valve positioned in the first fluid passageway formed in the base plate, wherein in the open state of the valve device, the umbrella valve is in a first position in the first fluid passageway in response to a force of fluid flowing from the first fluid passageway toward the fluid chamber, and fluid flows through at least one opening in a first flange portion of the umbrella valve and the first fluid passageway, through the fluid chamber, and out of the valve device through the second fluid passageway, and in the closed state of the valve device, the umbrella valve is in a second position in the first fluid passageway in which a second flange portion of the umbrella valve extends across the first opening to close the first opening in the base plate and block a flow of fluid between the fluid chamber and the first fluid passageway in response to a force of fluid flowing from the second fluid passageway toward the fluid chamber.

In some aspects, the techniques described herein relate to a fluid control system for an implantable fluid operated inflatable device, including a housing; at least one fluid passageway defined within the housing; and a valve device positioned in the at least one fluid passageway, the valve device including a base plate; a diaphragm coupled to the base plate; a fluid chamber defined between the base plate and the diaphragm; a first opening formed in the base plate, the first opening connecting the fluid chamber to a first fluid passageway; a second opening formed in the base plate, the second opening connecting the fluid chamber to a second fluid passageway; and a fluid control device provided at the one of the first opening or the second opening, wherein in an open state of the valve device, the first fluid passageway, the fluid chamber, and the second fluid passageway guides fluid through the valve device in a first fluid flow direction, and in a closed state of the valve device, the fluid control device is configured to close the one of the first fluid passageway or the second fluid passageway in response to a force applied in a second fluid flow direction, opposite the first fluid flow direction, to maintain the closed state of the valve device.

In some implementations, the fluid control device includes a spring check valve positioned in the second fluid passageway, wherein in the open state of the valve device, the spring check valve is opened in response to a force of fluid flowing from the fluid chamber to the second fluid passageway, and in the closed state of the valve device, the spring check valve is closed against the diaphragm in response to a force of fluid flowing from the second fluid passageway toward the fluid chamber.

In some implementations, the fluid control device includes a spring plate positioned on the base plate of the valve device, with the spring check valve formed in the spring plate, at a position corresponding to the second opening in the base plate and an opening formed at a position corresponding to the first opening in the base plate; and a foil plate positioned between the base plate and the spring plate, including a first opening at a position corresponding to the first opening in the base plate and the opening in the spring plate and a second opening corresponding to the spring check valve and the second opening in the base plate.

In some implementations, a dimension of a disc portion of the spring check valve is greater than a corresponding dimension of the second opening in the base plate, such that in the closed state the force exerted on the disc portion by fluid flowing from the second fluid passageway toward the fluid chamber maintains the spring check valve in a closed state against the second opening in the base plate.

In some implementations, the fluid control device includes a seal positioned on the base plate, at a position corresponding to the first opening in the base plate, wherein in the open state of the valve device, fluid flows through the valve device from the first fluid passageway, through a space in the fluid chamber between the seal and the diaphragm, and out of the valve device through the second fluid passageway, and in the closed state of the valve device, at least a portion of the seal deforms to maintain a seal against the diaphragm in response to a force of fluid flowing from the second fluid passageway toward the fluid chamber.

In some implementations, the seal includes a body portion; a first flange portion at a first end portion of the body portion, the first flange portion being coupled to the base plate, with an opening extending through the body portion aligned with the first opening and the first fluid passageway; a second flange portion at a second end portion of the body portion, wherein in the closed state of the valve device, the second flange portion is positioned against the diaphragm and forms a seal with the diaphragm, and the second flange portion is configured to deform so as to maintain the seal with the diaphragm in response to the force of fluid from the second fluid passageway exerted on the diaphragm and corresponding deformation of the diaphragm.

In some implementations, the fluid control device includes an umbrella valve positioned in the first fluid passageway formed in the base plate, wherein in the open state of the valve device, the umbrella valve is in a first position in the first fluid passageway in response to a force of fluid flowing from the first fluid passageway toward the fluid chamber, and fluid flows through the umbrella valve and the first fluid passageway, through the fluid chamber, and out of the valve device through the second fluid passageway, and in the closed state of the valve device, the umbrella valve is in a second position in the first fluid passageway that closes the first opening in the base plate to block a flow of fluid between the fluid chamber and the first fluid passageway in response to a force of fluid flowing from the second fluid passageway toward the fluid chamber.

In some implementations, the umbrella valve includes a body portion; a first flange portion at a first end portion of the body portion; at least one opening formed in the first flange portion, aligned with the first opening and the first fluid passageway; and a second flange portion at a second end portion of the body portion.

In some implementations, in the open state of the valve device, the umbrella valve is in the first position in the first fluid passageway, with the second flange portion positioned in the fluid chamber and spaced apart from a first side portion of the base plate to provide for fluid communication between the first fluid passageway and the fluid chamber via the first opening in the base plate, and the first flange portion is positioned at a second side portion of the base plate, with the at least one opening in the first flange portion aligned with the first fluid passageway; and in the closed state of the valve device, the umbrella valve is in the second position in the first fluid passageway, with the second flange portion positioned on the first side portion of the base plate, extending across the first opening in the base plate in response to the force of fluid from the second fluid passageway so as to maintain a closed state of the first fluid passageway and the closed state of the valve device.

In some implementations, the fluid control device includes a flap seal valve positioned in the first fluid passageway, wherein in the open state of the valve device, fluid flows through the valve device from the first fluid passageway, through an opening in the flap seal valve, through the fluid chamber, and out of the valve device through the second fluid passageway, and in the closed state of the valve device, at least a portion of the flap seal valve deforms to close the opening in response to a force of fluid flowing from the second fluid passageway toward the fluid chamber.

In some implementations, the flap seal valve includes a body portion; a base portion at a first end portion of the body portion, the base portion being fixed in the first fluid passageway, wherein an opening extends through the base portion to provide for fluid communication between the first fluid passageway and the fluid chamber; a flap portion at a second end portion of the body portion, wherein in the closed state of the valve device, the flap portion is configured to deform so as to close the opening in the base portion in response to the force of fluid from the second fluid passageway toward the fluid chamber.

In some aspects, the techniques described herein relate to an implantable fluid operated inflatable device, including a fluid control system configured to control fluid flow between a fluid reservoir and an inflatable member, the fluid control system including at least one fluid passageway defined within a housing; and a fluidic component positioned in the at least one fluid passageway and configured to provide for a flow of fluid in a first flow direction, and to restrict a flow of fluid in a second flow direction, the fluidic component including a fluid chamber defined between a base plate and a deformable diaphragm; and an auxiliary fluid control device provided at one of an inlet into the fluid chamber or an outlet of the fluid chamber, wherein in an open state of the fluidic component, the auxiliary fluid control device is in an open state such that inlet, the fluid chamber, and the outlet provide for fluid flow through the fluidic component in the first flow direction, and in a closed state of the fluidic component, the auxiliary fluid control device is configured to close one of the inlet or the outlet of the fluid chamber in response to a force applied in the second flow direction, to maintain the closed state of the fluidic component.

In some implementations, the auxiliary fluid control device includes a spring check valve positioned in the outlet, wherein in the open state of the fluidic component, the spring check valve is opened in response to a force of fluid, flowing from the fluid chamber toward the outlet, exerted on a disc portion of the spring check valve, and in the closed state of the fluidic component, the spring check valve is closed against a foil positioned between the spring check valve and the outlet in response to a force of fluid, flowing from the outlet toward the fluid chamber, exerted on the disc portion of the spring check valve.

In some implementations, a dimension of the disc portion of the spring check valve is greater than a corresponding dimension of an opening formed in the foil, such that in the closed state the force exerted on the disc portion maintains the spring check valve in the closed state.

In some implementations, the auxiliary fluid control device includes a seal positioned on the base plate, at a position corresponding to the inlet, the seal including a body portion; a first flange portion at a first end portion of the body portion; and a second flange portion at a second end portion of the body portion, wherein the first flange portion is coupled to the base plate, with an opening extending through the body portion aligned with the inlet, and the second flange portion is positioned in the fluid chamber; in the open state of the fluidic component, fluid flows from the inlet, through a space in the fluid chamber between the second flange portion and the diaphragm, and out of the fluidic component through the outlet; and in the closed state of the fluidic component, the second flange portion is positioned against the diaphragm and forms a seal with the diaphragm, and the second flange portion is configured to deform so as to maintain the seal with the diaphragm in response to the force of fluid from the outlet exerted on the diaphragm and a corresponding deformation of the diaphragm.

In some implementations, the auxiliary fluid control device includes an umbrella valve positioned in inlet, the umbrella valve including a body portion positioned in the inlet; a first flange portion at a first end portion of the body portion; at least one opening formed in the first flange portion, aligned with the inlet; and a second flange portion at a second end portion of the body portion, positioned in the fluid chamber, wherein in the open state of the fluidic component, the umbrella valve is in a first position in the inlet, in which the second flange portion is spaced apart from a first side portion of the base plate to provide for fluid communication between the inlet and the fluid chamber, and the first flange portion is positioned at a second side portion of the base plate, with the at least one opening in the first flange portion aligned with the inlet, such that fluid flows through the at least one opening, through the inlet into the fluid chamber and out of the fluidic component through the outlet; and in the closed state of the fluidic component, the umbrella valve is in a second position in the inlet, in which the second flange portion is positioned on the first side portion of the base plate, extending across the inlet in response to a force of fluid from the outlet so as to maintain a closed state of the inlet and the closed state of the fluidic component.

In some implementations, the auxiliary fluid control device includes a flap seal valve positioned in the inlet, the flap seal valve including a body portion; a base portion at a first end portion of the body portion, the base portion being fixed in the inlet, with an opening extending through the base portion to provide for fluid communication with the fluid chamber; and a flap portion at a second end portion of the body portion, wherein in the open state of the fluidic component, fluid flows through the opening in the base portion and into fluid chamber, and out of the fluidic component through the outlet, and in the closed state of the fluidic component, the flap portion is configured to deform so as to close the opening in the base portion in response to a force of fluid from the outlet toward the fluid chamber.

In some implementations, the first flow direction provides for the flow of fluid from the fluid reservoir to the inflatable member, and the second flow direction provides for the flow of fluid from the inflatable member to the fluid reservoir.

In some implementations, the inlet is defined by a first opening in the base plate providing for fluid communication between a first fluid passageway of the fluidic component and the fluid chamber; and the outlet is defined by a second opening in the base plate providing for fluid communication between the fluid chamber and a second fluid passageway of the fluidic component.

In implementations, the fluidic component is one of a valve device, a pump device, or a combined pump and valve device.

### BRIEF DESCRIPTION OF THE DRAWINGS

FIG. 1 is a block diagram of an implantable fluid operated inflatable device.

FIG. 2 illustrates a system including an example implantable fluid operated inflatable device.

FIG. 3A is a schematic diagram of a fluidic architecture of an implantable fluid operated inflatable device.

FIG. 3B is a schematic diagram of a fluidic architecture of an implantable fluid operated inflatable device.

FIG. 4A is an exploded view of an example valve device of a fluid control system of a fluid operated inflatable device.

FIG. 4B is a cross-sectional view of the example valve device shown in FIG. 4A, in a closed position.

FIG. 4C is a cross-sectional view of the example valve device shown in FIG. 4A, in an open position.

FIG. 4D is a top view of the example valve device shown in FIG. 4A.

FIG. 5A is a schematic view of an example valve device including an example auxiliary flow control device, with the example valve device in an open position.

FIG. 5B is a schematic view of an example valve device including an example auxiliary flow control device, with the example valve device in a closed position.

FIG. 6A illustrates an example auxiliary flow control device, in an open position.

FIG. 6B illustrates the example auxiliary flow control device shown in FIG. 6A, in a closed position.

FIG. 6C is an exploded perspective view of the example auxiliary flow control device shown in FIGS. 6A and 6B relative to a base plate of an example valve device.

FIG. 6D is an exploded perspective view of an example auxiliary flow control device relative to a base plate of an example valve device.

FIG. 7A illustrates an example valve device including an example auxiliary flow control device, in an open position.

FIG. 7B illustrates the example valve device including the example auxiliary flow control device shown in FIG. 7A, in a closed position.

FIG. 7C is a perspective view of the example auxiliary flow control device shown in FIGS. 7A and 7B.

FIG. 8A illustrates an example valve device including an example auxiliary flow control device, in an open position.

FIG. 8B illustrates the example valve device including the example auxiliary flow control device shown in FIG. 8A, in a closed position.

FIG. 8C is a perspective view of the example auxiliary flow control device shown in FIGS. 8A and 8B.

FIG. 9A illustrates an example valve device including an example auxiliary flow control device, in an open position.

FIG. 9B illustrates the example valve device including the example auxiliary flow control device shown in FIG. 9A, in a closed position.

FIG. 9C is a perspective view of the example auxiliary flow control device shown in FIGS. 9A and 9B.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

An implantable fluid operated inflatable device may include a fluid control system. In some examples, the fluid control system includes at least one pump and/or at least one valve and/or at least one combined pump and valve device. In some examples, the components of the fluid control system control the flow of fluid between a fluid reservoir and an inflatable member of the implantable fluid operated inflatable device, to provide for the inflation/pressurization and deflation/depressurization of the inflatable member. In some situations, the fluid control system may experience fluctuations in pressure, including spikes in pressure. For example, in some situations, the pumps and/or valves of the fluid control system may experience pressure fluctuations and/or spikes in pressure that exceed typical design pressures. In some situations, these types of pressure fluctuations, or spikes, may result in an unintended opening of a pump or valve, and/or unintended deflation/depressurization of the inflatable member, and/or unintended inflation/pressurization of the inflatable member. A fluid control system, in accordance with implementations described herein, includes a fluid control device including a check valve that prevents the unintentional opening of a fluid passageway in response to a spike in pressure experienced within the fluid control system. In some examples, the fluid control device is one of a pumping device, a valve device, or a combined pump and valve device. In some examples, the check valve is positioned in an outlet flow port of the fluid control device. In some examples, the check valve is positioned in an inlet flow port of the fluid control device. In some examples, the check valve includes a spiral check valve positioned in a fluid passageway of the fluid control device. In some examples, the check valve includes a spool shaped O-ring positioned in a fluid passageway of the fluid control device. In some examples, the check valve includes an umbrella valve positioned in a fluid passageway of the fluid control device.

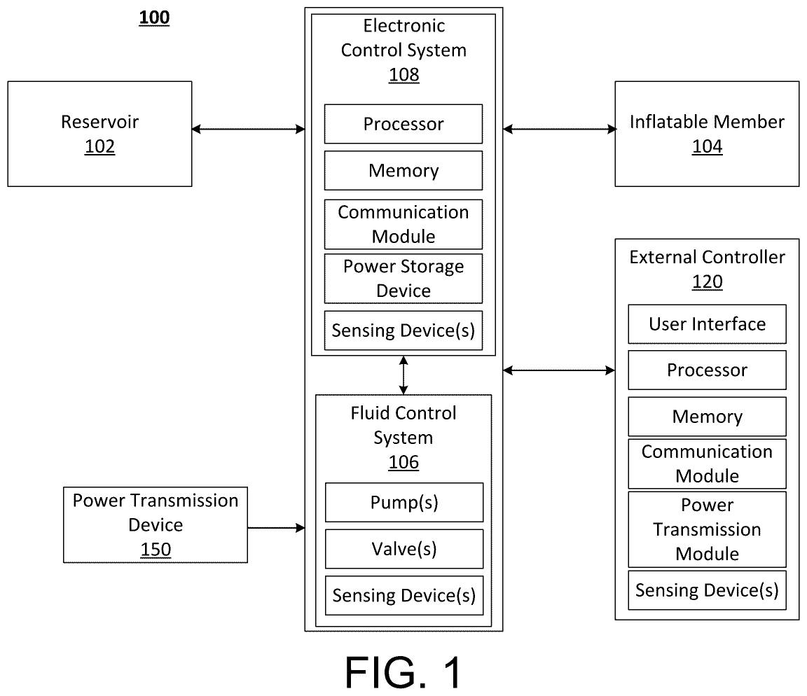

FIG. 1 is a block diagram of an example implantable fluid operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidics components such as one or more pumps, one or more valves and the like configured to transfer fluid between the fluid reservoir 102 and the inflatable member 104. The fluid control system 106 can include one or more sensing devices that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid operated inflatable device 100 and/or communication with one or more external device(s). In some examples, the electronic control system 108 includes components such as a processor, a memory, a communication module, a power storage device, or battery, sensing devices such as, for example an accelerometer, and other such components configured to provide for the operation and control of the implantable fluid operated inflatable device 100. In some examples, the communication module of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in or incorporated into a housing. In some implementations, at least a portion of the electronic control system 108 is physically separate from the fluid control system 106. In some implementations, some modules of the electronic control system 108 are coupled to or incorporated into the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid operated inflatable device 100. In some implementations, at least some aspects of the operation of the implantable fluid operated inflatable device 100 may be manually controlled.

In some examples, electronic monitoring and control of the implantable fluid operated inflatable device 100 may provide for improved patient control of the device, improved patient comfort, improved patient safety, and the like. In some examples, electronic monitoring and control of the implantable fluid operated inflatable device 100 may afford the opportunity for tailoring of the operation of the inflatable device 100 by a physician without further surgical intervention. Fluidic architecture defining the flow and control of fluid through the implantable fluid operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

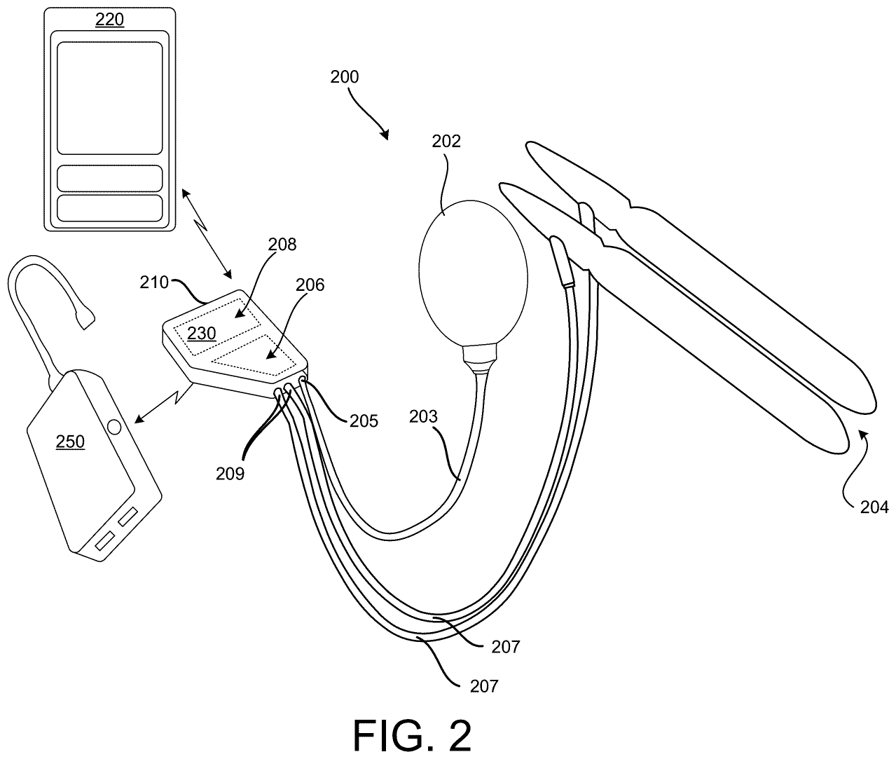

The example implantable fluid operated inflatable device 100 may be representative of a number of different types of implantable fluid operated devices. For example, the implantable fluid operated inflatable device 100 shown in FIG. 1 may be representative of an inflatable penile prosthesis as shown in FIG. 2. In some implementations, the example implantable fluid operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid operated inflatable device 200 in the form of an example inflatable penile prosthesis is shown in FIG. 2. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2, the inflatable member 204 is in the form of a pair of inflatable cylinders. In the example shown in FIG. 2, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the example shown in FIG. 2, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 can be charged and/or recharged by a power transmission module of the external controller 220, and/or by a power transmission device 250, that is separate from the external controller 220.

The principles to be described herein are applicable to the example implantable fluid operated inflatable device, in the form of the example inflatable penile prostheses shown in FIG. 2, and other types of implantable fluid operated inflatable devices that rely on a pump and valve assembly including various fluidics components to provide for the transfer of fluid between the different fluid filled implantable components to achieve inflation, deflation, pressurization, depressurization, deactivation, occlusion, and the like for effective operation. The example implantable fluid operated inflatable device 200 shown in FIG. 2 includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control of the inflatable device 200, improved accuracy in operation of the inflatable device 200, improved patient comfort, improved patient safety, and the like. In some situations, this improved control and improved accuracy in the operation of the inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and/or valve devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices and/or combined pump and valve devices within a fluid circuit of the pump assembly to control the transfer fluid between the fluid reservoir and the inflatable member. In some examples, the pump assembly including the one or more pump devices and valve device(s) and/or combined pump and valve devices is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices and/or combined pump and valve devices include piezoelectric elements. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid operated device to provide for patient safety and device efficacy.

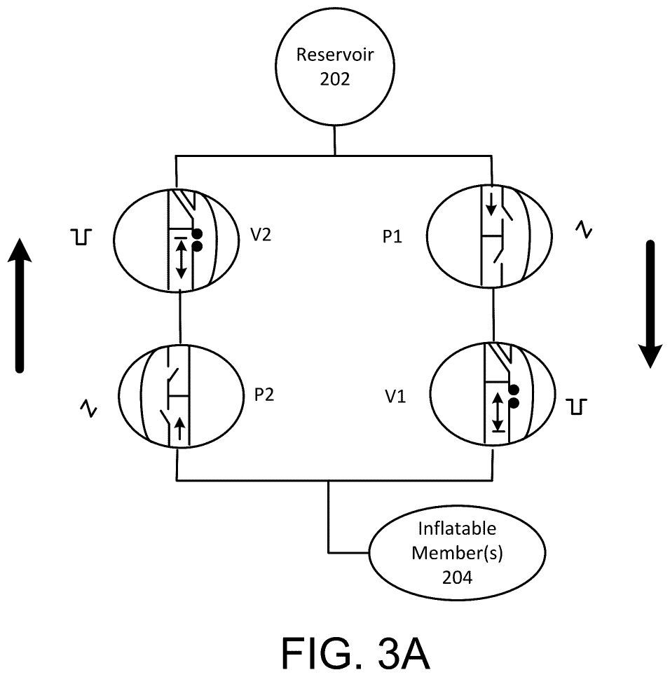

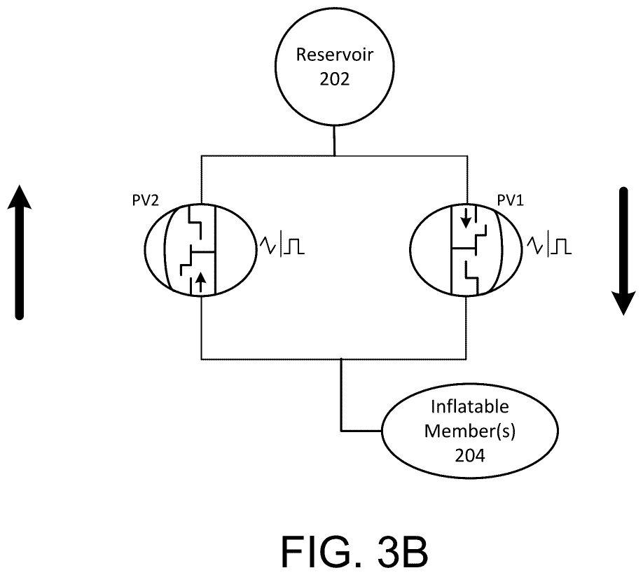

FIG. 3A is a schematic diagram of an example fluidic architecture for an implantable fluid operated inflatable device, according to an aspect. FIG. 3B is a schematic diagram of an example fluidic architecture for an implantable fluid operated inflatable device, according to an aspect. The fluidic architecture of an implantable fluid operated inflatable device can include other arrangements of fluidic channels, pump(s)/valve(s), pressure sensor(s) and other components than the examples shown in FIGS. 3A and 3B.

The example fluidic architecture shown in FIG. 3A includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3A includes a second pump P2 and a second valve V2 positioned in a second fluid passageway, between the inflatable member 204 and the reservoir 202, to control the flow of fluid from the inflatable member 204 to the reservoir 202. As shown in FIG. 3B, in some examples, the first pump and the first valve are included in a combination pump and valve device PV1 provided in the first fluid passageway, and the second pump and the second valve are included in a second combination pump and valve device PV2 provided in the second fluid passageway.

In example fluidic architecture shown in FIG. 3A, the first pump P1 and the first valve V1 operate to pump fluid from the reservoir 202 to the inflatable member 204 through the first fluid passageway to provide for inflation of the inflatable member 204, while the second valve V2 closes the second fluid passageway to provide to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

In the example arrangement shown in FIG. 3B, the first combined pump and valve device PV1 and the second combined pump and valve device PV2 may be operated in a first mode to inflate or pressurize the inflatable member 204, and in a second mode to deflate or repressurize the inflatable member 204. In the first mode of operation, the first combined pump and valve device PV1 convey fluid from the reservoir 202 to the inflatable member 204, while the second combined pump and valve device PV2 remains closed/inoperable to prevent flow of fluid from the inflatable member 204 towards the reservoir 202 to prevent deflation/depressurization. The first combined pump and valve device PV1 may remain operable to pump fluid to the inflatable member 204 until a desired pressure is achieved. The first combined pump and valve device PV1 may be closed once the desired pressure is achieved, to maintain the inflatable member 204 at the desired pressure/inflated state. In the second mode of operation, the second combined pump and valve device PV2 convey fluid from the inflatable member 204 to the reservoir 202, while the first combined pump and valve device PV1 remains closed/inoperable to prevent flow of fluid from the reservoir 202 towards the inflatable member 204 to prevent inflation/pressurization. The second combined pump and valve device PV2 may remain operable to pump fluid to the reservoir 202 until a desired pressure is achieved at the inflatable member 204. The second combined pump and valve device PV2 may be closed once the desired pressure is achieved, to maintain the inflatable member 204 at the desired pressure/in the deflated state.

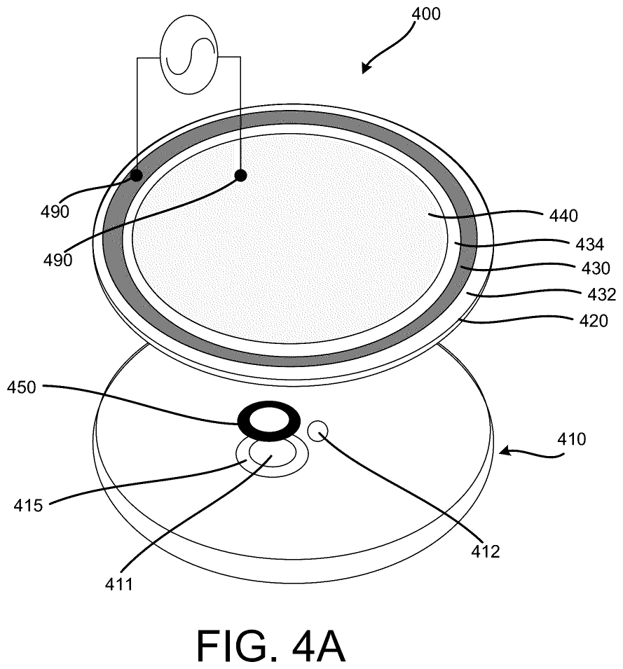

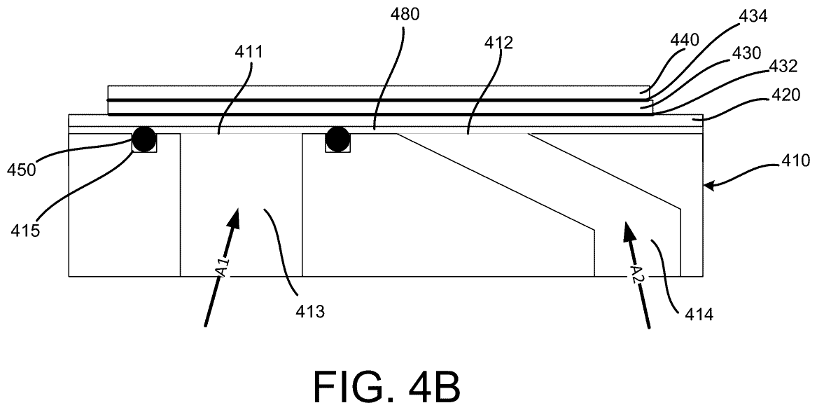

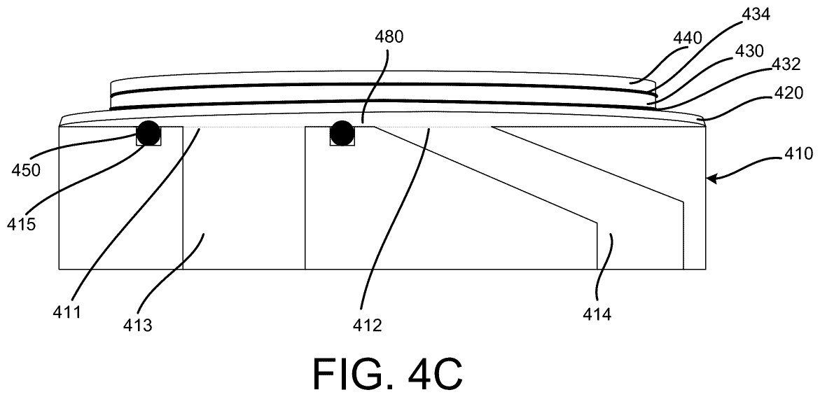

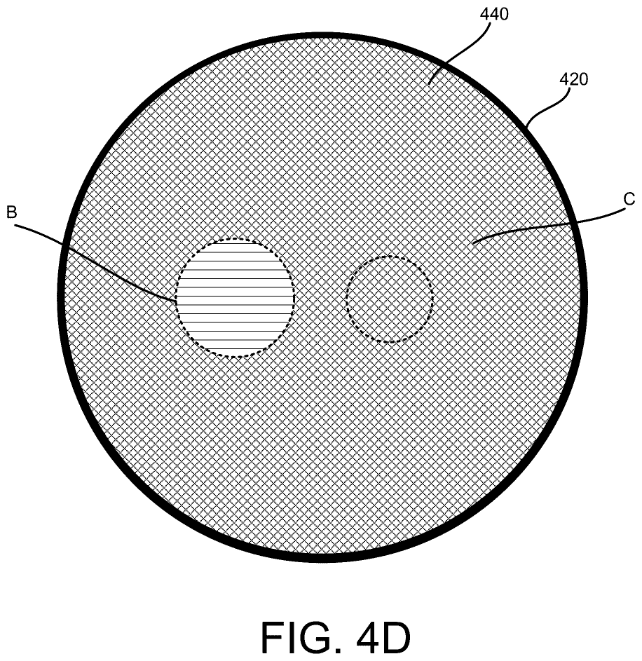

FIG. 4A is a partially exploded perspective view of an example valve device 400. FIGS. 4B and 4C are cross-sectional views of the example valve device 400 shown in FIG. 4A, in an assembled state. FIG. 4D is a schematic top view of the example valve device 400. The example valve device 400 shown in FIGS. 4A-4C may be an example of a fluid control device, or a fluidic component, included in the fluid control system 206 of the example electronically controlled fluid manifold 230 described above.

In the example arrangement shown in FIGS. 4A-4C, the example valve device 400 includes a base plate 410 defining a base portion of the valve device 400. A diaphragm 420 is positioned on the base plate 410. A piezoelectric element 440 is positioned on the diaphragm 420, with an isolation layer 430 positioned between the diaphragm 420 and the piezoelectric element 440. In some examples, an epoxy layer 432 provides for the coupling of the isolation layer 430 on the diaphragm 420. In some examples, an epoxy layer 434 provides for the coupling of the piezoelectric element 440 on the isolation layer 430. In some examples, one or more electrodes 490 are arranged on the example valve device 400. In the example shown in FIG. 4A, the example valve device 400 includes a pair of electrodes 490 coupled between the isolation layer 430 and the piezoelectric element 440. Application of a voltage to the piezoelectric element 440 causes a deflection or deformation of the piezoelectric element 440, and a corresponding deflection or deformation of the diaphragm 420 and the isolation layer 430 coupled thereto. Application of voltage in this manner causes the valve device 400 to move between the closed position shown in FIG. 4B and the open position shown in FIG. 4C. In some examples, an alternating application of voltage to the piezoelectric element 440 causes the valve device 400 to alternate between the closed position and the open position, such that the valve device 400 can also perform a pumping function.

In the example arrangement shown in FIGS. 4A-4C, a fluid chamber 480 is defined between the base plate 410 and the diaphragm 420. The base plate 410 includes a first opening 411 that provides for communication between a first fluid passageway 413 and the fluid chamber 480. The base plate 410 includes a second opening 412 that provides for communication between a second fluid passageway 414 and the fluid chamber 480. In the example arrangement shown in FIGS. 4A-4C, the base plate 410 includes a recess 415 surrounding the first opening 411, with a seal 450, in the form of an O-ring in the example shown in FIGS. 4A-4C, fitted in the recess 415. In some examples, a top portion of the seal 450 is pressed against the diaphragm 420 in the closed position of the valve device 400, as shown in FIG. 4B to close off the chamber 480, and inhibit the flow of fluid through the example valve device 400, between the first fluid passageway 413 and the second fluid passageway 414 via the chamber 480. In some examples, in which the valve device 400 does not include a seal 450 as shown in FIGS. 4A-4C, the diaphragm 420 is seated against the base plate 410 to close off the chamber 480 and inhibit the flow of fluid through the valve device 400. In the open position of the example valve device 400, the base plate 410 and the top portion of the seal 450 are separated, or spaced apart from the diaphragm 420 due to the deflection of the diaphragm 420. This positioning of the seal 450 and the base plate 410 relative to the diaphragm 420 opens the chamber 480, and allows fluid to flow through the example valve device 400, between the first fluid passageway 413 and the second fluid passageway 414 via the fluid chamber 480.

In some situations, the fluid control system 206, including fluid control devices such as the example valve device 400 and/or other such fluid control devices, may experience fluctuations, or spikes in pressure. In some situations, the fluctuations, or spikes in pressure may be greater than a pressure applied by the piezoelectric element 440 (and isolation layer 430 and diaphragm 420 coupled thereto) on the seal 450 and/or the base plate 410 to maintain a closed state of the valve device 400. For example, pressure applied in the direction of the arrow A1 (via the first fluid passageway 413 and first opening 411) and/or in the direction of the arrow A2 (via the second fluid passageway 414 and second opening 412) may exert a pressure on the piezoelectric element 440/isolation layer 430/diaphragm 420 that is greater than the closing force exerted on the seal 450 and/or base plate 410 by the piezoelectric element 440/isolation layer 430/diaphragm 420. In some examples, the force A1 may exert a pressure on the piezoelectric element 440/isolation layer 430/diaphragm 420 in a pressure area A shown in FIG. 4D. In some examples, the force A2 may exert a pressure on the piezoelectric element 440/isolation layer 430/diaphragm 420 in a pressure area B shown in FIG. 4D.

Forces, or pressure exerted on the piezoelectric element 440/isolation layer 430/diaphragm 420 in this manner can cause an unintentional opening of the valve device 400. This unintentional opening of the valve device 400 may, in turn, cause unintentional deflation/depressurization of the inflatable member 204, or unintentional inflation/pressurization of the inflatable member 204, depending on a direction of flow through the valve device 400 (or other fluid control device of the fluid control system 206).

In some implementations, a fluid control device of the fluid control system 206, such as the example valve device 400, includes a check valve, or a one-way valve, positioned in a fluid passageway of the fluid control device. In some examples, the check valve is positioned in a portion of the fluid passageway so as to inhibit the unintended flow of fluid through the fluid control device in the event of a fluctuation, or spike in pressure. In some examples, the check valve is positioned in the fluid passageway so as to counteract a back pressure that would otherwise overcome the closing pressure and cause unintentional flow through the device. In some examples, a check valve is positioned at the second opening 412 in the base plate 410, defining an interface between the second fluid passageway 414 and the chamber 480 of the example valve device 400. In some examples, a check valve is positioned at the first opening 411 in the base plate 410, defining an interface between the first fluid passageway 413 and the chamber 480 of the example valve device 400.

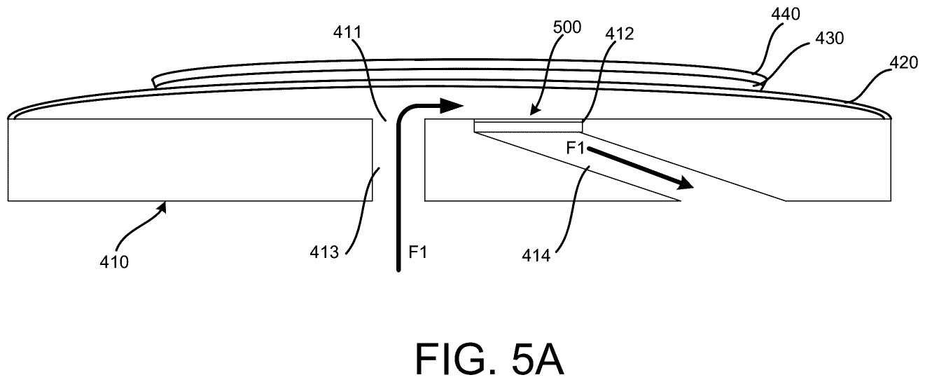

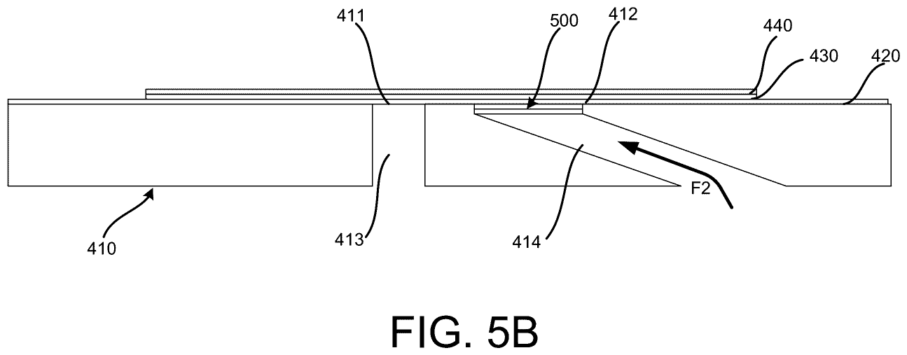

FIGS. 5A and 5B are cross-sectional views of the example valve device 400 shown in FIGS. 4A-4D, including an example flow control device 500 positioned in one of the fluid passageways of the example valve device 400.

FIG. 5A illustrates an example in which the valve device 400 is open, allowing fluid to flow in the direction of the arrows F1, through the first fluid passageway 413, into the chamber 480, and out of the valve device 400 through the second fluid passageway 414. The example shown in FIG. 5A may illustrate an open position of the valve device 400 that allows fluid to flow, for example, from the reservoir 202 to the inflatable member 204 to provide for inflation/pressurization of the inflatable member 204.

In the example arrangement shown in FIGS. 5A and 5B, the example flow control device 500 is positioned at the second opening 412 formed in the base plate 410, the second opening 412 providing for fluid communication between the fluid chamber 480 and the second fluid passageway 414. In some examples, the flow control device 500 is a check valve, or a one-way valve, that allows for flow in one direction (in this example, in the direction of the arrows F1), while inhibiting flow in the opposite direction.

FIG. 5B illustrates the closed position of the valve device 400, in which the flow of fluid through the valve device 400 is blocked. In some examples, the closed position shown in FIG. 5B may maintain an inflation pressure of the inflatable member 204. As described above, in some situations, pressure fluctuations and/or pressure spikes may exert a force, or pressure on the valve device 400 in the closed position. FIG. 5B illustrates a pressure spike, or a back pressure, exerted in the direction of the arrow F2. In the example described above with respect to FIGS. 4A-4D, this type of pressure spike, or back pressure exerted on the diaphragm 420/piezoelectric element 440 could cause an unintentional opening of the valve device 400, and an unintentional deflation/depressurization of the inflatable member 204. In the example shown in FIG. 5B, the flow control device 500 (positioned at the second opening 412, between the second fluid passageway 414 and the fluid chamber 480), for example, in the form of a check valve or a one-way valve, remains in the closed position in response to the pressure spike/back pressure/flow of fluid in the direction of the arrow F2. Thus, the positioning of the flow control device 500 at the second opening 412, allowing flow in a first direction, i.e., the direction of the arrows F1, while blocking flow in a second direction, i.e., the direction of the arrow F2, maintains the closed state of the valve device 400, even in response to fluctuation in pressure, or pressure spike, or back pressure.

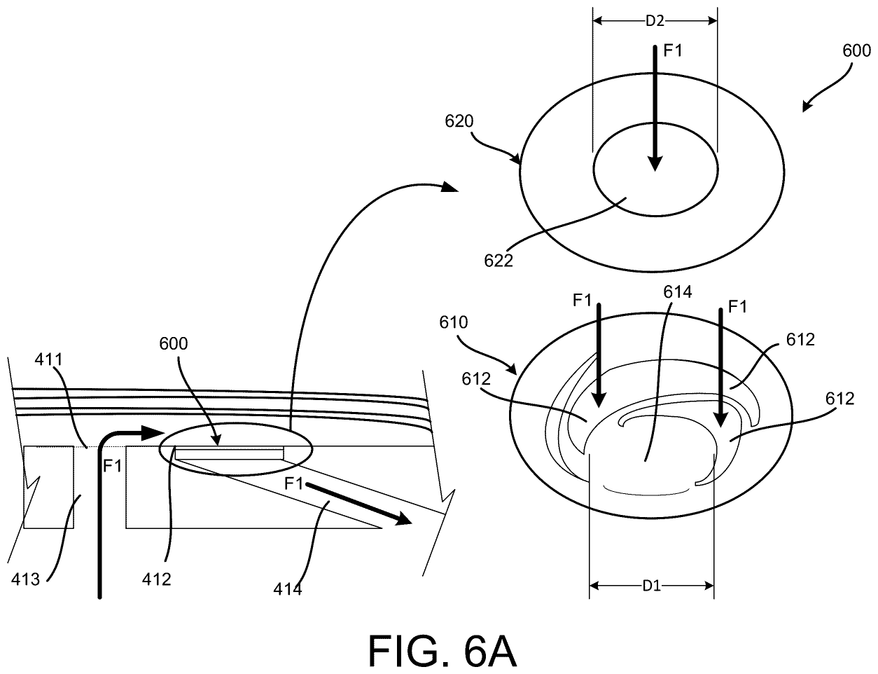

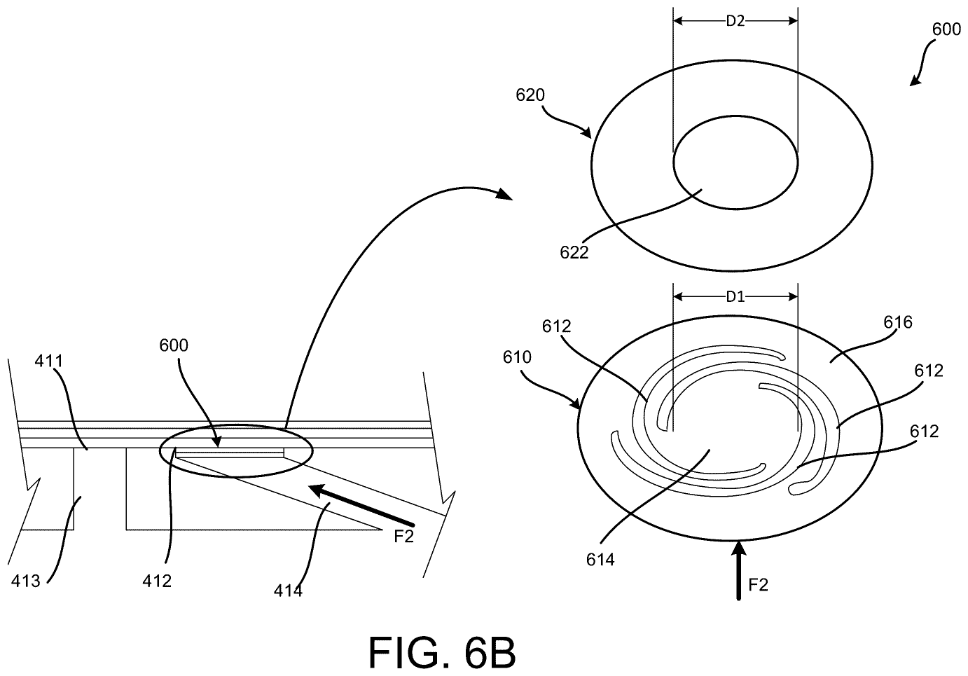

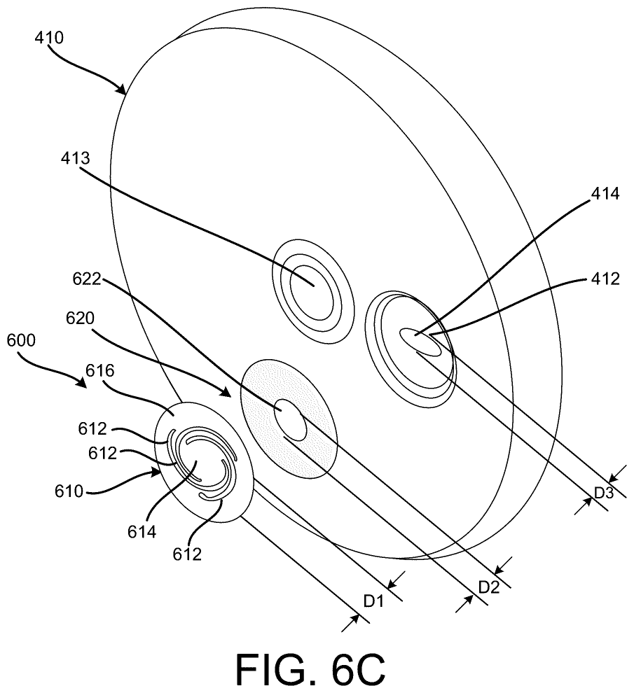

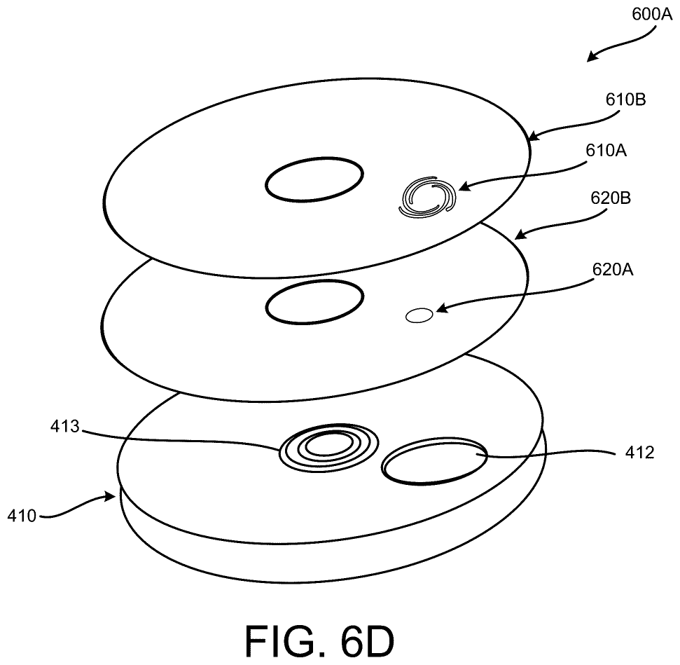

FIGS. 6A-6C illustrate an example auxiliary flow control device 600 that can be incorporated into the example valve device 400, for example, in the manner described above with respect to FIGS. 5A and 5B. In particular, the example auxiliary flow control device 600 may be a check valve, or a one-way valve, that provides for flow in one direction, and that inhibits flow in a second direction.

FIG. 6A illustrates the example flow control device 600 in the example valve device 400, in the open position. FIG. 6B illustrates the example flow control device 600 in the example valve device 400, in the closed position. FIG. 6C is an exploded perspective view of the example valve device 400 relative to the base plate 410 of the example valve device 400.

The example flow control device 600 shown in FIGS. 6A and 6B includes a spring check valve 610 positioned against a foil 620. In the open position of the flow control device 600 (corresponding, for example, to the open position of the valve device 400 shown in FIGS. 4C and 5A), fluid can flow in the direction of the arrows F1, through the flow control device 600. That is, a pressure of the fluid flowing from the fluid chamber 480 through the second opening 412 in the base plate 410 and an opening 622 in the foil 620 (for example, an opening 622 aligned with the second opening 412 in the base plate 410) exerts a pressure on a disc portion 614 of the spring check valve 610. The pressure, or force exerted on the disc portion 614 of the spring check valve 610 moves the disc portion 614 in the direction of the arrows F1. This movement of the disc portion 614 separates the disc portion 614 from a rim portion 616 of the spring check valve 610, so that fluid can flow through openings defined by slots 612 formed in the spring check valve 610 in the open, or expanded position shown in FIG. 6A.

In the closed position of the flow control device 600 (corresponding, for example, to the closed position of the valve device 400 shown in FIGS. 4B and 5B), the flow of fluid from the fluid chamber 480 into the second fluid passageway 414 is blocked by the position of the diaphragm 420 (and isolation layer 430/piezoelectric element 440) extending across the second opening 412 in the base plate 410. In the event of a spike in pressure, or force, or back pressure in the direction of the arrow F2, the flow control device 600 may maintain a closed position, or a closed state of the valve device 400. That is, the flow control device 600, in the form of a check valve or a one-way valve positioned in the second opening 412 in the base plate 410, may remain closed, even in the event of a spike in pressure, or back pressure, thus maintaining the closed state of the valve device 400.

For example, as shown in FIG. 6B, a dimension, for example, a diameter D1 of the disc portion 614 of the spring check valve 610 may be greater than a corresponding dimension, for example, a diameter D2 of the opening 622 of the foil 620. Thus, an overlap, or interference of an outer peripheral portion of the disc portion 614 of the spring check valve 610 with an inner peripheral portion of the opening 622 in the foil 620, thus restricting movement of the disc portion 614 of the spring check valve 610 through the opening 622, and closing the flow control device 600 and restricting flow from the second fluid passageway 414 into the fluid chamber 480.

The example flow control device 600 has been described with respect to the spring check valve 610 in combination with the foil 620 in FIGS. 6A-6C. The principles described can be similarly applied to the use of the spring check valve 610, without the foil 620, positioned in the second opening 412 in the base plate 410. That is, in a configuration of the flow control device 600 including just the spring check valve 610, movement of the disc portion 614 of the spring check valve 610 in the direction of the arrow F2 may be restricted by a position of the disc portion 614 against the diaphragm 420. In some examples, the diameter D1 of the disc portion 614 of the spring check valve 610 may be greater than a corresponding dimension D3 of a corresponding portion of the second opening 412 in which the flow control device 600 is installed. In this case, an overlap, or interference of the outer peripheral portion of the disc portion 614 of the spring check valve 610 with an inner peripheral portion of the second opening 412 in the base plate 410, thus restricting movement of the disc portion 614 of the spring check valve 610 through the second opening 412, thus maintaining the closed position of the flow control device 600.

FIG. 6D is an exploded perspective view, illustrating an example auxiliary flow control device 600A that can be incorporated into the example valve device 400. In particular, the example auxiliary flow control device 600A may include check valve, or a one-way valve, that provides for flow in one direction, and that inhibits flow in a second direction.

In the example arrangement shown in FIG. 6D, the example flow control device 600A includes a spring check valve 610A incorporated into a spring plate 610B, and a foil 620A incorporated into a foil plate 620B, with the foil plate 620B positioned between the spring plate 610B and the base plate 410 of the example valve device 400. The spring check valve 610A incorporated into the spring plate 610B, and the foil 620A incorporated into the foil plate 620B, may function similarly to the spring check valve 610 and foil 620 described above with respect to FIGS. 6A-6C, providing for resistance to spikes in pressure, or backpressure, during operation of the example valve device 400, particularly in the closed state of the example valve device 400. Similarly, in some examples, the spring check valve 610A incorporated into the spring plate 610B may be operable without the foil plate 620B to provide for resistance to spikes in pressure, or backpressure, during operation of the example valve device 400, particularly in the closed state of the example valve device 400.

In the examples presented above with respect to FIGS. 6A-6D, operation of the example valve device 400 including the example flow control device 600 positioned in the second opening 412 of the base plate 410 is described in an example arrangement in which fluid flows into the example valve device 400 via the first fluid passageway 413, and out of the example valve device 400 via the second fluid passageway 414, to, for example, provide for the flow of fluid from the reservoir 202 to the inflatable member 204, simply for purposes of discussion and illustration. The principles described above are similarly applicable to an arrangement in which the flow control device 600 is positioned in the first opening 411/first fluid passageway 413 (not explicitly shown in FIGS. 6A-6D), and in which fluid flows into the example valve device 400 via the second fluid passageway 414, and out of the example valve device 400 via the first fluid passageway 413 to, for example, provide for the flow of fluid from the inflatable member 204 to the reservoir 202. The principles described above are similarly applicable to an arrangement in which the flow control device 600 is positioned in the second opening 412/second fluid passageway 413, with spring 610/spring plate 610B positioned between the foil 620/foil plate 620B and the base plate 410 (not explicitly shown in FIGS. 6A-6D), and in which fluid flows into the example valve device 400 via the second fluid passageway 414, and out of the example valve device 400 via the first fluid passageway 413 to, for example, provide for the flow of fluid from the inflatable member 204 to the reservoir 202.

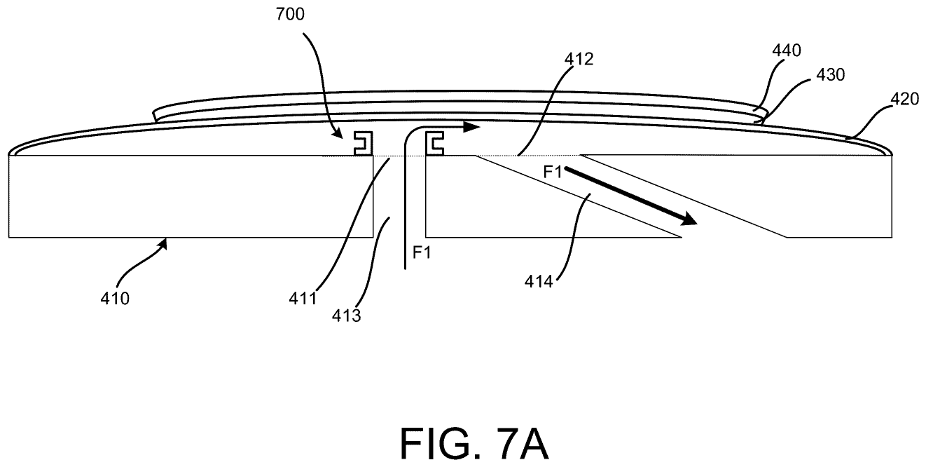

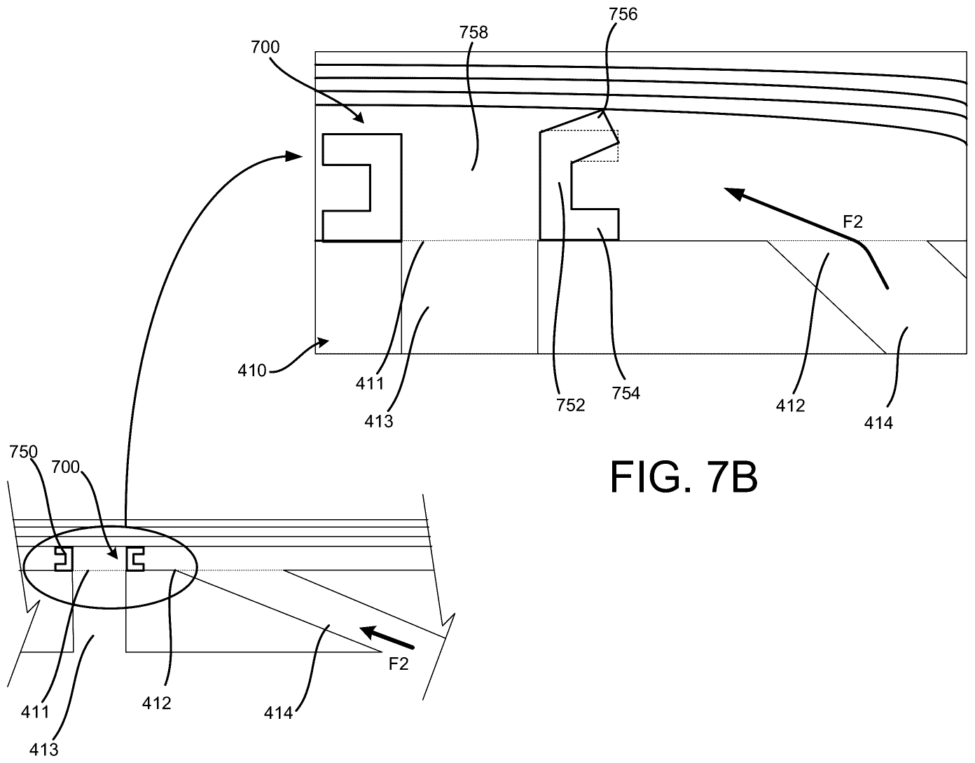

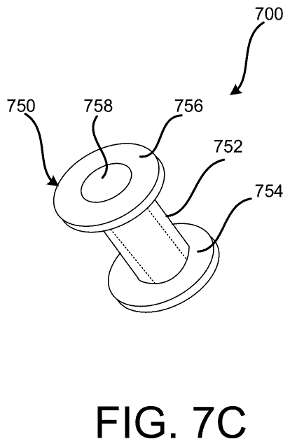

FIGS. 7A-7C illustrate an example auxiliary flow control device 700 that can be incorporated into the example valve device 400. In the example arrangement shown in FIGS. 7A-7C, the example auxiliary flow control device 700 is in the form of a seal 750 positioned at the first opening 411 formed in the base plate 410, at the interface between the first fluid passageway 413 and the fluid chamber 480 of the example valve device 400. The example flow control device 700 may allow for flow in one direction, and inhibit flow in a second direction.

FIG. 7A illustrates the example flow control device 700 in the example valve device 400, in the open position. FIG. 7B illustrates the example flow control device 700 in the example valve device 400, in the closed position. FIG. 7C is a perspective view of the example flow control device 700, including features of a seal 750 defining the flow control device 700.

The seal 750 includes a body portion 752, with a first flange portion 754 extending outward, for example radially outward, from a first end portion of the body portion 752, and a second flange portion 756 extending outward, for example, radially outward, from a second end portion of the body portion 752. An opening 758 is defined in the body portion 752, extending through the seal 750. In some examples, the seal 750 is made of an elastomer material, or a compliant material, that allows for some amount of flex or movement in response to the application of force or pressure.

In the arrangement shown in FIGS. 7A and 7B, the first flange portion 754 of the seal 750 is fixed to the base plate 410, at a position corresponding to the first opening 411/first fluid passageway 413. The opening 758 in the seal 750 is aligned with the first opening 411/first fluid passageway 413 so that, in the open state of the valve device 400, fluid from the first fluid passageway 413 can flow through the first opening 411, through the opening 758 of the seal 750, through the fluid chamber 480, and out through the second opening 412/second fluid passageway 414 in the direction of the arrows F1, as shown in FIG. 7A.

In the closed position of the valve device 400 shown in FIG. 7B, the diaphragm 420 is positioned against, for example, pressed against, the second flange portion 756 of the seal 750. In this arrangement, the second flange portion 756 defines a sealing portion of the seal 750, with the pressure exerted on the second flange portion 756 by the diaphragm 420 forming a seal that blocks or inhibits the flow of fluid. That is, in the example arrangement shown in FIG. 7B, flow of fluid from the first fluid passageway 413 into and through the fluid chamber 480 is blocked or inhibited by the positioning of the diaphragm 420 against the second flange portion 756, and the seal formed therebetween.

In some examples, a spike in pressure, or force, or back pressure in the direction of the arrow F2 may cause the diaphragm 420 (and piezoelectric element 440/isolation layer 430) to deflect or deform, unintentionally opening a portion of the fluid chamber 480 with the valve device 400 in the closed state, as shown in the enlarged portion of FIG. 7B. In response to this spike in pressure, or force, or back pressure and corresponding flow of fluid in the direction of the arrow F2, while the valve device 400 is in the closed position, at least a portion of the seal 750 may deflect, or deform, to maintain the sealed condition of the fluid chamber 480, and restrict flow through the fluid chamber 480 in the closed state of the valve device 400, as shown in the enlarged portion of FIG. 7B. In particular, in the example shown in FIG. 7B, the force of the fluid exerted on the seal 750 in the direction of the arrow F2 causes the second flange portion 756 to deflect from an at rest position (shown in dashed lines) to a deflected position. This deflection maintains the seal between the flow control device 700 including the seal 750 and the diaphragm 420, and maintains the closed state of the valve device 400. Thus, the flow control device 700, in the form of the seal 750 positioned at the first opening 411 in the base plate 410, may deform or deflect to maintain a closed state of the fluid chamber 480 and a corresponding closed state of the valve device 400, even in the event of a fluctuation or spike in pressure, or back pressure.

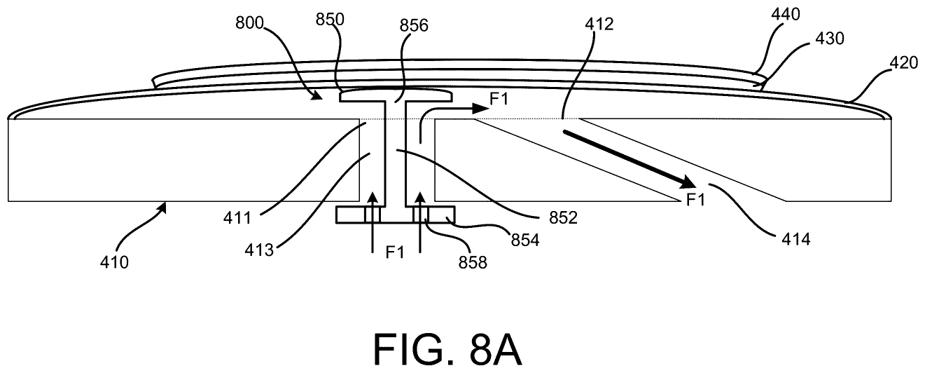

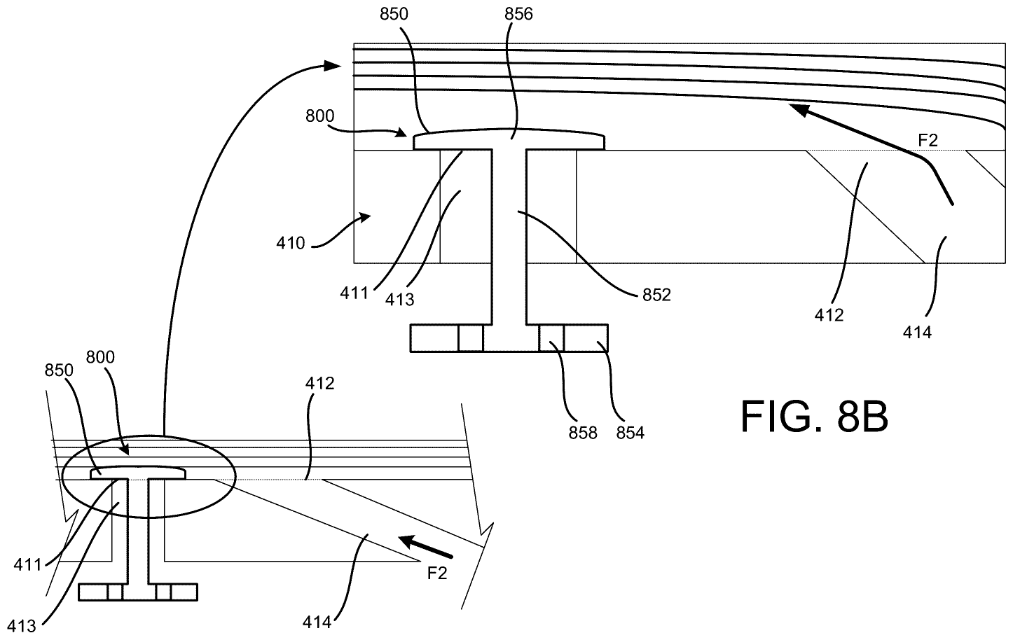

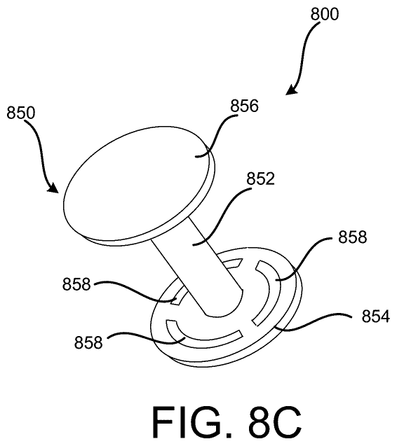

FIGS. 8A-8C illustrate an example auxiliary flow control device 800 that can be incorporated into the example valve device 400. In the example arrangement shown in FIGS. 8A-8C, the example auxiliary flow control device 800 is in the form of an umbrella valve 850 positioned at the first opening 411 formed in the base plate 410, at the interface between the first fluid passageway 413 and the fluid chamber 480 of the example valve device 400. The example flow control device 800 may allow for flow in one direction, and inhibit flow in a second direction.

FIG. 8A illustrates the example flow control device 800 in the example valve device 400, in the open position. FIG. 8B illustrates the example flow control device 800 in the example valve device 400, in the closed position. FIG. 8C is a perspective view of the example flow control device 800, in the form of the umbrella valve 850.

The example umbrella valve 850 includes a body portion 852, with a first flange portion 854 extending outward, for example radially outward, from a first end portion of the body portion 852, and a second flange portion 856 extending outward, for example, radially outward, from a second end portion of the body portion 852. At least one opening 858 is defined in the first flange portion 854.

In the arrangement shown in FIGS. 8A and 8B, the flow control device 800, in the form of the umbrella valve 850, floats in the first opening 411/first fluid passageway 413 of the base plate 410. The at least one opening 858 in the first flange portion 854 of the umbrella valve 850 is aligned with the first fluid passageway 413/first opening 411, to allow fluid to flow through the at least one opening 858 and into the first fluid passageway 413. For example, in the open state of the valve device 400 shown in FIG. 8A, a force of the fluid, flowing in the direction of the arrows F1 moves the umbrella valve 850 upward (in the example orientation shown in FIG. 8A), so that the first flange portion 854 is moved away from or spaced apart from the first opening 411. This allows fluid to flow through the at least one opening 858 in the first flange portion 854 into the first fluid passageway 413, through the fluid chamber 480 and into the second fluid passageway 414 in the direction of the arrows F1. Thus, the force of the fluid, flowing in the direction of the arrows F1, causes the umbrella valve 850 to move from an at rest position, in an upward direction, to the position shown in FIG. 8A, allowing fluid to flow into and through the valve device 400 in the direction of the arrows F1.

In the closed position of the valve device 400 shown in FIG. 8B, the diaphragm 420 (and the isolation layer 430/piezoelectric element 440 coupled thereto) is in an at rest, un-deflected, or un-deformed state, and the second flange portion 856 of the umbrella valve 850 is positioned so as to close the first opening 411 of the base plate 410. In the arrangement shown in FIG. 8B, the flow of fluid is thus blocked between first fluid passageway 413 and the fluid chamber 480.

In some examples, a spike in pressure, or force, or back pressure in the direction of the arrow F2 may cause the diaphragm 420 (and piezoelectric element 440/isolation layer 430) to deflect or deform, as shown in the enlarged portion of FIG. 8B. This deformation of the diaphragm 420 may allow fluid to flow from the second fluid passageway 414 into the fluid chamber 480 through the second opening 412. In the example shown in FIG. 8B, the first opening 411 remains covered, or blocked by the second flange portion 856 of the umbrella valve 850. This positioning of the second flange portion 856 across the first opening 411 prevents fluid from flowing from the fluid chamber 480 into the second fluid passageway 414 in response to the spike in pressure in the direction of the arrow F2, and unintentional opening of the valve device 400. Thus, the flow control device 800, in the form of the umbrella valve 850 positioned at the first opening 411 in the base plate 410, may maintain a closed state of the fluid chamber 480 and a corresponding closed state of the valve device 400, even in the event of a fluctuation or spike in pressure, or back pressure.

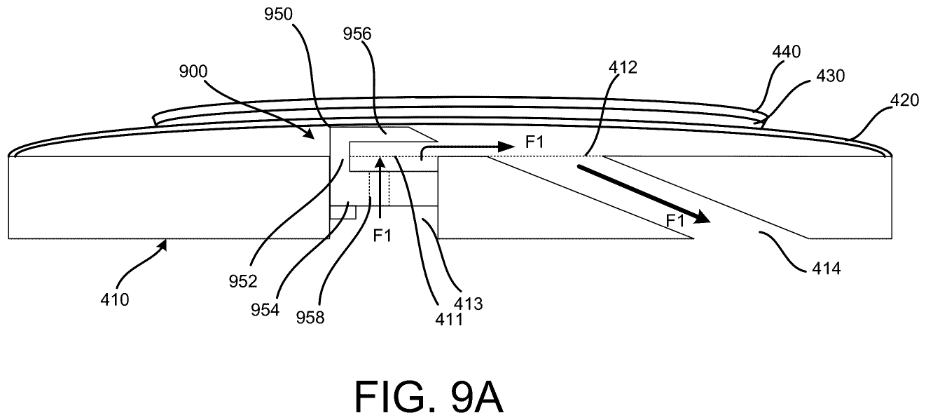

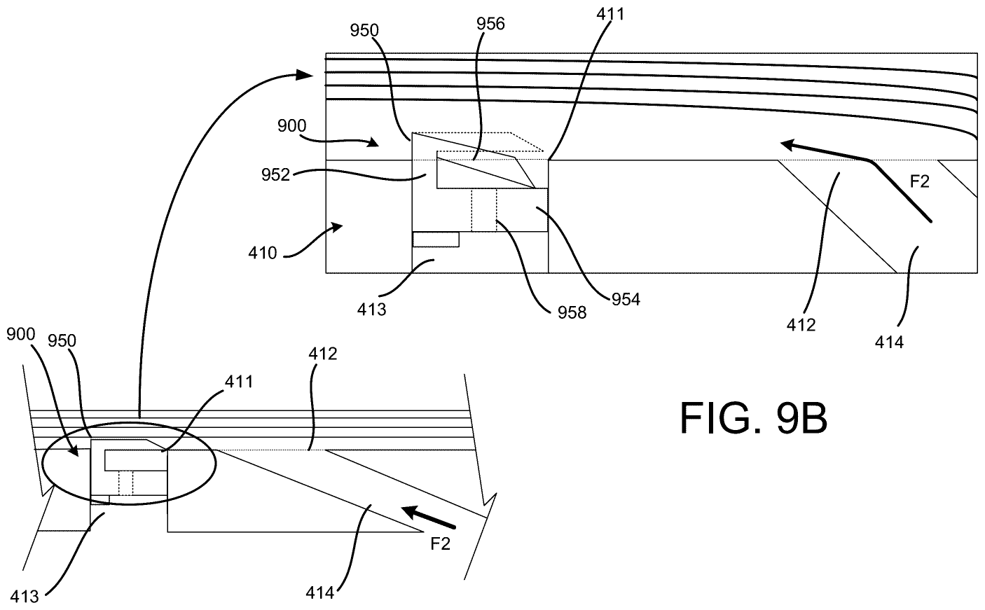

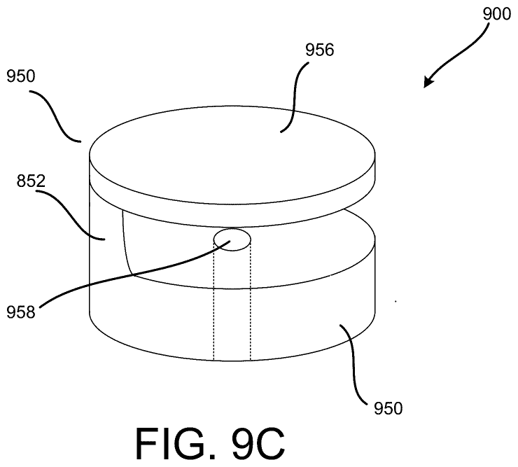

FIGS. 9A-9C illustrate an example auxiliary flow control device 900 that can be incorporated into the example valve device 400. In the example arrangement shown in FIGS. 9A-9C, the example auxiliary flow control device 900 is in the form of flap seal valve 950 positioned at the first opening 411 formed in the base plate 410, at the interface between the first fluid passageway 413 and the fluid chamber 480 of the example valve device 400. The example flow control device 900 may allow for flow in one direction, and inhibit flow in a second direction.

FIG. 9A illustrates the example flow control device 900 in the example valve device 400, in the open position. FIG. 9B illustrates the example flow control device 900 in the example valve device 400, in the closed position.

The example flap seal valve 950 includes a body portion 952, with a base portion 954 a first end portion of the body portion 952, and a flap portion 956 at a second end portion of the body portion 952. At least one opening 958 is defined in base portion 954. In some examples, at least a portion of the flap seal valve 950 is made of an elastomer material, or a compliant material, that allows for some amount of flex or movement in response to the application of force or pressure. In some examples, at least the flap portion 956 of the flap seal valve 950 is made of an elastomer material, or a compliant material that allow for flexure of at least a portion of the flap seal valve 950. In the arrangement shown in FIGS. 9A and 9B, the flow control device 900, in the form of the flap seal valve 950, the base portion 954 may be fixed in the first fluid passageway 413, while the flap portion 956 remains movable.

In the open state of the valve device 400 shown in FIG. 9A, the flap portion 956 is separated from, or spaced apart from the base portion 954, leaving the at least one opening 958 open to the fluid chamber 480. In the open state of the valve device 400 shown in FIG. 9A, fluid from the first fluid passageway 413 flows into the fluid chamber 480 through the at least one opening 958, and out of the valve device 400 through the second fluid passageway 414, in the direction of the arrows F1.

In the closed position of the valve device 400 shown in FIG. 9B, the diaphragm 420 (and the isolation layer 430/piezoelectric element 440 coupled thereto) is in an at rest, un-deflected, or un-deformed state, and the flap portion 956 of the flap seal valve 950 is positioned so as to close the first opening 411 of the base plate 410. In the arrangement shown in FIG. 9B, the flow of fluid is thus blocked between first fluid passageway 413 and the fluid chamber 480.

In some examples, a spike in pressure, or force, or back pressure in the direction of the arrow F2 may cause the diaphragm 420 (and piezoelectric element 440/isolation layer 430) to deflect or deform, as shown in the enlarged portion of FIG. 9B. This deformation of the diaphragm 420 may allow fluid to flow from the second fluid passageway 414 into the fluid chamber 480 through the second opening 412. In response to this spike in pressure, or force, or back pressure and corresponding flow of fluid in the direction of the arrow F2, while the valve device 400 is in the closed position, at least a portion of the flap seal valve 950 may deflect, or deform, to maintain the sealed condition of the fluid chamber 480, and restrict flow through the fluid chamber 480 in the closed state of the valve device 400, as shown in the enlarged portion of FIG. 9B. In particular, in the example shown in FIG. 9B, the force of the fluid exerted on the flap seal valve 950 in the direction of the arrow F2 causes the flap portion 956 to deflect from an at rest position (shown in dashed lines) to a deflected position. This deflection of the flap portion 956 blocks the at least one opening 958 in the base portion 954, and thus blocks the flow of fluid from the fluid chamber 480 to the first fluid passageway 413. Thus, the flow control device 900, in the form of the flap seal valve 950 positioned at the first opening 411 in the base plate 410, may deform or deflect to maintain a closed state of the fluid chamber 480 and a corresponding closed state of the valve device 400, even in the event of a fluctuation or spike in pressure, or back pressure.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A fluid control system for an implantable fluid operated inflatable device, comprising:
a housing;
at least one fluid passageway defined within the housing; and
a valve device positioned in the at least one fluid passageway, the valve device including:
a base plate;
a diaphragm coupled to the base plate;
a fluid chamber defined between the base plate and the diaphragm;
a first opening formed in the base plate, the first opening connecting the fluid chamber to a first fluid passageway;
a second opening formed in the base plate, the second opening connecting the fluid chamber to a second fluid passageway; and
a fluid control device provided at the one of the first opening or the second opening, wherein
in an open state of the valve device, the first fluid passageway, the fluid chamber, and the second fluid passageway guides fluid through the valve device in a first fluid flow direction, and
in a closed state of the valve device, the fluid control device is configured to close the one of the first fluid passageway or the second fluid passageway in response to a force applied in a second fluid flow direction, opposite the first fluid flow direction, to maintain the closed state of the valve device.

2. The fluid control system of claim 1, wherein the fluid control device comprises a spring check valve positioned in the second fluid passageway, wherein
in the open state of the valve device, the spring check valve is opened in response to a force of fluid flowing from the fluid chamber to the second fluid passageway, and
in the closed state of the valve device, the spring check valve is closed against the diaphragm in response to a force of fluid flowing from the second fluid passageway toward the fluid chamber.

3. The fluid control system of claim 2, wherein the fluid control device includes:
a spring plate positioned on the base plate of the valve device, with the spring check valve formed in the spring plate, at a position corresponding to the second opening in the base plate and an opening formed at a position corresponding to the first opening in the base plate; and
a foil plate positioned between the base plate and the spring plate, including a first opening at a position corresponding to the first opening in the base plate and the opening in the spring plate and a second opening corresponding to the spring check valve and the second opening in the base plate.

4. The fluid control system of claim 2, wherein a dimension of a disc portion of the spring check valve is greater than a corresponding dimension of the second opening in the base plate, such that in the closed state the force exerted on the disc portion by fluid flowing from the second fluid passageway toward the fluid chamber maintains the spring check valve in a closed state against the second opening in the base plate.

5. The fluid control system of claim 1, wherein the fluid control device comprises a seal positioned on the base plate, at a position corresponding to the first opening in the base plate, wherein
in the open state of the valve device, fluid flows through the valve device from the first fluid passageway, through a space in the fluid chamber between the seal and the diaphragm, and out of the valve device through the second fluid passageway, and
in the closed state of the valve device, at least a portion of the seal deforms to maintain a seal against the diaphragm in response to a force of fluid flowing from the second fluid passageway toward the fluid chamber.

6. The fluid control system of claim 5, wherein the seal includes:
a body portion;
a first flange portion at a first end portion of the body portion, the first flange portion being coupled to the base plate, with an opening extending through the body portion aligned with the first opening and the first fluid passageway;
a second flange portion at a second end portion of the body portion, wherein
in the closed state of the valve device,
the second flange portion is positioned against the diaphragm and forms a seal with the diaphragm, and
the second flange portion is configured to deform so as to maintain the seal with the diaphragm in response to the force of fluid from the second fluid passageway exerted on the diaphragm and corresponding deformation of the diaphragm.

7. The fluid control system of claim 1, wherein the fluid control device comprises an umbrella valve positioned in the first fluid passageway formed in the base plate, wherein
in the open state of the valve device, the umbrella valve is in a first position in the first fluid passageway in response to a force of fluid flowing from the first fluid passageway toward the fluid chamber, and fluid flows through the umbrella valve and the first fluid passageway, through the fluid chamber, and out of the valve device through the second fluid passageway, and
in the closed state of the valve device, the umbrella valve is in a second position in the first fluid passageway that closes the first opening in the base plate to block a flow of fluid between the fluid chamber and the first fluid passageway in response to a force of fluid flowing from the second fluid passageway toward the fluid chamber.

8. The fluid control system of claim 7, wherein the umbrella valve includes:
a body portion;
a first flange portion at a first end portion of the body portion;
at least one opening formed in the first flange portion, aligned with the first opening and the first fluid passageway; and
a second flange portion at a second end portion of the body portion.

9. The fluid control system of claim 8, wherein
in the open state of the valve device,
the umbrella valve is in the first position in the first fluid passageway, with
the second flange portion positioned in the fluid chamber and spaced apart from a first side portion of the base plate to provide for fluid communication between the first fluid passageway and the fluid chamber via the first opening in the base plate, and
the first flange portion is positioned at a second side portion of the base plate, with the at least one opening in the first flange portion aligned with the first fluid passageway; and
in the closed state of the valve device,
the umbrella valve is in the second position in the first fluid passageway, with
the second flange portion positioned on the first side portion of the base plate, extending across the first opening in the base plate in response to the force of fluid from the second fluid passageway so as to maintain a closed state of the first fluid passageway and the closed state of the valve device.

10. The fluid control system of claim 1, wherein the fluid control device comprises a flap seal valve positioned in the first fluid passageway, wherein
in the open state of the valve device, fluid flows through the valve device from the first fluid passageway, through an opening in the flap seal valve, through the fluid chamber, and out of the valve device through the second fluid passageway, and
in the closed state of the valve device, at least a portion of the flap seal valve deforms to close the opening in response to a force of fluid flowing from the second fluid passageway toward the fluid chamber.

11. The fluid control system of claim 10, wherein the flap seal valve includes:
a body portion;
a base portion at a first end portion of the body portion, the base portion being fixed in the first fluid passageway, wherein an opening extends through the base portion to provide for fluid communication between the first fluid passageway and the fluid chamber;
a flap portion at a second end portion of the body portion, wherein
in the closed state of the valve device,
the flap portion is configured to deform so as to close the opening in the base portion in response to the force of fluid from the second fluid passageway toward the fluid chamber.

12. An implantable fluid operated inflatable device, comprising:
a fluid control system configured to control fluid flow between a fluid reservoir and an inflatable member, the fluid control system including:
at least one fluid passageway defined within a housing; and
a fluidic component positioned in the at least one fluid passageway and configured to provide for a flow of fluid in a first flow direction, and to restrict a flow of fluid in a second flow direction, the fluidic component including:
a fluid chamber defined between a base plate and a deformable diaphragm; and
an auxiliary fluid control device provided at one of an inlet into the fluid chamber or an outlet of the fluid chamber, wherein
in an open state of the fluidic component, the auxiliary fluid control device is in an open state such that inlet, the fluid chamber, and the outlet provide for fluid flow through the fluidic component in the first flow direction, and
in a closed state of the fluidic component, the auxiliary fluid control device is configured to close one of the inlet or the outlet of the fluid chamber in response to a force applied in the second flow direction, to maintain the closed state of the fluidic component.

13. The implantable fluid operated inflatable device of claim 12, wherein the auxiliary fluid control device comprises a spring check valve positioned in the outlet, wherein
in the open state of the fluidic component, the spring check valve is opened in response to a force of fluid, flowing from the fluid chamber toward the outlet, exerted on a disc portion of the spring check valve, and
in the closed state of the fluidic component, the spring check valve is closed against a foil positioned between the spring check valve and the outlet in response to a force of fluid, flowing from the outlet toward the fluid chamber, exerted on the disc portion of the spring check valve.

14. The implantable fluid operated inflatable device of claim 13, wherein a dimension of the disc portion of the spring check valve is greater than a corresponding dimension of an opening formed in the foil, such that in the closed state the force exerted on the disc portion maintains the spring check valve in the closed state.

15. The implantable fluid operated inflatable device of claim 12, wherein the auxiliary fluid control device comprises a seal positioned on the base plate, at a position corresponding to the inlet, the seal including:
a body portion;
a first flange portion at a first end portion of the body portion; and
a second flange portion at a second end portion of the body portion, wherein
the first flange portion is coupled to the base plate, with an opening extending through the body portion aligned with the inlet, and the second flange portion is positioned in the fluid chamber;
in the open state of the fluidic component, fluid flows from the inlet, through a space in the fluid chamber between the second flange portion and the diaphragm, and out of the fluidic component through the outlet; and
in the closed state of the fluidic component,
the second flange portion is positioned against the diaphragm and forms a seal with the diaphragm, and
the second flange portion is configured to deform so as to maintain the seal with the diaphragm in response to the force of fluid from the outlet exerted on the diaphragm and a corresponding deformation of the diaphragm.

16. The implantable fluid operated inflatable device of claim 12, wherein the auxiliary fluid control device comprises an umbrella valve positioned in inlet, the umbrella valve including:
a body portion positioned in the inlet;
a first flange portion at a first end portion of the body portion;
at least one opening formed in the first flange portion, aligned with the inlet; and
a second flange portion at a second end portion of the body portion, positioned in the fluid chamber, wherein
in the open state of the fluidic component,
the umbrella valve is in a first position in the inlet, in which
the second flange portion is spaced apart from a first side portion of the base plate to provide for fluid communication between the inlet and the fluid chamber, and
the first flange portion is positioned at a second side portion of the base plate, with the at least one opening in the first flange portion aligned with the inlet, such that fluid flows through the at least one opening, through the inlet into the fluid chamber and out of the fluidic component through the outlet; and
in the closed state of the fluidic component,
the umbrella valve is in a second position in the inlet, in which the second flange portion is positioned on the first side portion of the base plate, extending across the inlet in response to a force of fluid from the outlet so as to maintain a closed state of the inlet and the closed state of the fluidic component.

17. The implantable fluid operated inflatable device of claim 12, wherein the auxiliary fluid control device comprises a flap seal valve positioned in the inlet, the flap seal valve including:
a body portion;
a base portion at a first end portion of the body portion, the base portion being fixed in the inlet, with an opening extending through the base portion to provide for fluid communication with the fluid chamber; and
a flap portion at a second end portion of the body portion, wherein
in the open state of the fluidic component,
fluid flows through the opening in the base portion and into fluid chamber, and out of the fluidic component through the outlet, and
in the closed state of the fluidic component,
the flap portion is configured to deform so as to close the opening in the base portion in response to a force of fluid from the outlet toward the fluid chamber.

18. The implantable fluid operated inflatable device of claim 12, wherein the first flow direction provides for the flow of fluid from the fluid reservoir to the inflatable member, and the second flow direction provides for the flow of fluid from the inflatable member to the fluid reservoir.

19. The implantable fluid operated inflatable device of claim 12, wherein
the inlet is defined by a first opening in the base plate providing for fluid communication between a first fluid passageway of the fluidic component and the fluid chamber; and
the outlet is defined by a second opening in the base plate providing for fluid communication between the fluid chamber and a second fluid passageway of the fluidic component.

20. The implantable fluid operated inflatable device of claim 12, wherein the fluidic component is one of a valve device, a pump device, or a combined pump and valve device.
